(19) United States
(12) Patent Application Publication
(10) Pub. No.: US 2004/0002747 A1
(43) Pub. Date: Jan. 1, 2004

(54) Device and method to expand treatment array

(75) Inventors: Thomas P. Ryan, Flemington, NJ (US); Alexander J. Sinton, Doylestown, PA (US)

Correspondence Address: SELITTO, BEHR & KIM, A PROFESSIONAL CORPORATION, PATENT & TRADEMARK ATTORNEYS, 203 MAIN STREET, METUCHEN, NJ, 08840, US

(73) Assignee: Ethicon, Inc.

(21) Appl. No.: 10/186,389

(22) Filed: Jun. 28, 2002

(51) Int. Cl. A61F 2/00

(52) U.S. Cl. 607/101

(57) ABSTRACT

A medical device is provided and includes an elongated tube that has a longitudinal axis and an expandable member attached to the tube. The expandable member is expandable from a collapsed configuration to an expanded configuration. A plurality of electrodes is attached to the expandable member. The electrodes are used to emit energy. Each of the electrodes is arranged generally coaxially relative to the tube when the expandable member is in its collapsed and expanded configurations.

### FIELD OF THE INVENTION

The present invention relates to an RF (radio frequency) device for use in the performance of RF thermal treatment of tissue, and more particularly, to an RF device adapted for use during a laparoscopic or percutaneous procedure.

### BACKGROUND OF THE INVENTION

During an RF procedure, a probe is placed into a target tissue for treating malignant and nonmalignant conditions. The probe is typically provided with an array of electrodes so that RF energy can be supplied to the target tissue.

Various RF devices have been employed to treat a large volume of tissue with a single applicator in a single procedure. One such device employs an array of wire electrodes that deploys and assumes an inverted umbrella shape after reaching the target tissue. When deployed, the inverted umbrella shape of the electrode array may be altered by tissue mechanical resistance or calcifications such that the electrode array exhibits a non-uniform pattern of thermal treatment. Such a non-uniform pattern is undesirable because tissue effects will be affected by the changing distance between adjacent electrodes that diverge or converge.

An alternative approach involves the use of an array of electrodes that have a large diameter. Such an approach is also undesirable because the large electrode array cannot be inserted through the body unless an open surgery is performed. Further, the large electrode array cannot be accommodated through a laparoscopic trocar that has a small diameter of 5 to 10 mm.

Accordingly, there is a need for an improved RF device having electrodes that deploy in a uniform and parallel manner along the length of the electrodes and that can be accommodated in a laparoscopic trocar having a small diameter.

### SUMMARY OF THE INVENTION

In accordance with the present invention, there is provided a medical device used for thermal treatment of tissue. The medical device includes an elongated tube that has a longitudinal axis and an expandable member attached to the tube. The expandable member is expandable from a collapsed configuration to an expanded configuration. A plurality of energy-emitting electrodes is attached to the expandable member. Each of the electrodes is arranged generally coaxially relative to the tube when the expandable member is in its collapsed and expanded configurations.

A method is also disclosed for performing thermal treatment of tissue using the medical device. Initially, the tube is placed near the tissue area with the expandable member in its collapsed configuration. In this step, the electrodes are in close proximity to each other and all of the electrodes are arranged generally co-axially relative to the tube. Then, the expandable member is expanded so that it assumes its expanded configuration. In this step, the electrodes are spaced apart from each other and all of the electrodes are arranged generally coaxially relative to the tube.

Other features and aspects of the present invention will become more fully apparent from the following detailed description of the exemplary embodiment, the appended claims and the accompanying drawings.

### BRIEF DESCRIPTION OF THE DRAWINGS

For a more complete understanding of the present invention, reference is made to the following detailed description of the exemplary embodiment considered in conjunction with the accompanying drawings, in which:

FIG. 1 is a front, perspective view of an RF device constructed in accordance with the present invention, which shows a multi-lumen tube in a retracted position;

FIG. 2 is a view similar to the view shown in FIG. 1, except that the multi-lumen tube is between its retracted and extended positions, and which shows a balloon member in its collapsed configuration;

FIG. 3 is a view similar to the view shown in FIG. 1, except that the multi-lumen tube is in its extended position and the balloon member is in its expanded configuration;

FIG. 4 is a cross-sectional view, taken along section lines IV-IV and looking in the direction of the arrows, of the RF device of FIG. 3; and

FIG. 5 is a cross-sectional view, taken along section lines V-V and looking in the direction of the arrows, of the RF device of FIG. 3.

### DETAILED DESCRIPTION OF THE EXEMPLARY EMBODIMENT

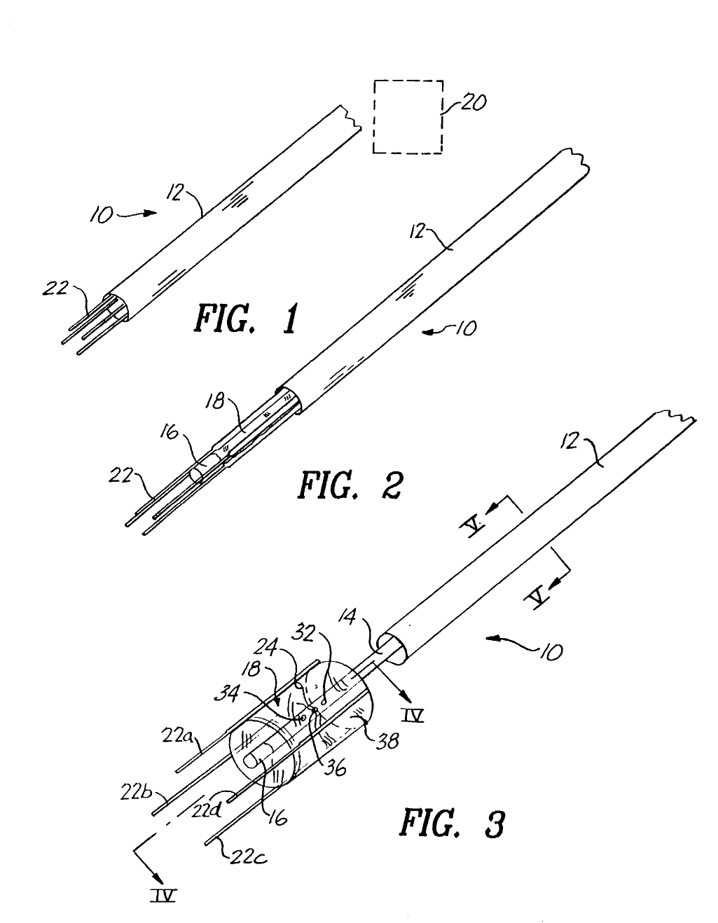

FIG. 1 shows an RF device 10 used for thermal treatment of tissue. The RF device 10 can be applied through 5-10 mm or larger laparoscopic trocar. Alternatively, the RF device 10 can be applied percutaneously to the affected tissue area.

With reference to FIGS. 1 to 3, the RF device 10 includes an outer sheath 12 which is linearly shaped and an elongated multi-lumen tube 14 sized and shaped to be coaxially received within the outer sheath 12. The multi-lumen tube 14 has a longitudinal axis and is also linearly shaped. The multi-lumen tube 14 is also sized and shaped to move relative to the outer sheath 12 by conventional methods such as by laparoscopic surgical tools (e.g., graspers, forceps, retractors) sliding through a plastic or metal trocar. As shown in FIG. 2, the multi-lumen tube 14 has a distal end 16 that can extend from the outer sheath 12. More particularly, the multi-lumen tube 14 is movable between a retracted position (see FIG. 1), in which the multi-lumen tube 14 retracts into the outer sheath 12, and an extended position (see FIG. 3), in which the multi-lumen tube 14 extends from the outer sheath 12. Alternatively, the outer sheath 12 can be sized and shaped to move relative to the multi-lumen tube 14.

Referring to FIG. 3, the RF device 10 further includes a balloon member 18 attached to the multi-lumen tube 14. The tube 14 extends completely through the balloon member 18. More particularly, the balloon member 18 is positioned along an intermediate length of the tube 14. For reasons to be discussed hereinafter, the balloon member 18 is sized and shaped to inflate into a fully expanded configuration as shown in FIG. 3 and to deflate into a fully collapsed configuration as shown in FIG. 2. Further, when the multi-lumen tube 14 is in its retracted position, the balloon member 18 is in its collapsed configuration and compressed within the outer sheath 12 so as to facilitate insertion into a trocar for delivery to the affected tissue area. When the multi-lumen tube 14 is in its extended position, the balloon member 18 is released from the outer sheath 12 such that it can move between its expanded configuration and collapsed configuration.

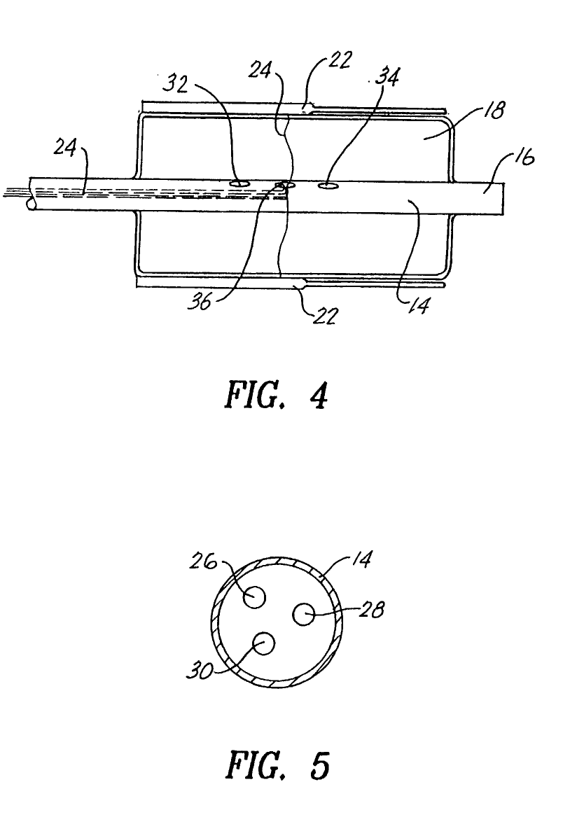

The RF device 10 is powered by an RF energy source, such as a conventional electrosurgical generator 20 (shown in phantom in FIG. 1). The operating frequency ranges from 300 to 1,000 kHz. As illustrated in FIG. 3, the RF device 10 includes a plurality of generally linearly shaped electrode needles 22 for delivering RF energy. The electrode diameters are between 0.25 and 1.0 mm, preferably 0.5 mm. The tips may be beveled, as a hypodermic needle, or other sharp tip. The polarity of the electrode needles 22 can be regulated such that each of the electrode needles 22 can be activated in various arrangements to be used as an active or return electrode. For example, two adjacent electrode needles 22a, 22b may be active and the remaining two electrode needles 22c, 22d may be return. Alternatively, adjacent electrode needles 22a, 22b may be active and return, making an alternating pattern. Alternatively, all electrode needles 22a, 22b, 22c, and 22d may be active and the return is a ground pad on the patient (not shown). Each of the electrode needles 22 is attached to the outer surface of the balloon member 18 by conventional attaching means, such as a solvent-based glue. It will be understood that although four electrode needles 22 are shown in FIG. 3, the number of electrode needles 22 can vary. The electrosurgical generator 20 is electrically connected to the electrode needles 22 and provides monopolar or bipolar energy to them in order to thermally treat tissue. With reference to FIG. 4, a plurality of wire leads 24 extends through the multi-lumen tube 14 and is electrically connected to the electrosurgical generator 20 and to the electrode needles 22. As shown in FIG. 4, each of the electrode needles 22 is attached to one of the wire leads 24.

When the balloon member 18 is in its collapsed configuration, the electrode needles 22 are in a compressed position, in which the electrode needles 22 are proximate to the multi-lumen tube 14 as shown in FIG. 2 so as to facilitate insertion within the outer sheath 22. Further, as the balloon member 18 inflates to its expanded configuration, the electrode needles 22 move to a deployed configuration, in which the electrode needles 22 move radially outward relative to the multi-lumen tube 14 such that each of the electrode needles 22 extends in a substantially parallel relationship relative to the other electrode needles 22 as shown in FIG. 3. The balloon member 18 expands to a diameter of between 10 and 50 mm, preferably 20 mm if four electrode needles 22a, 22b, 22c, 22d are provided and 30 mm if six electrode needles are provided. After extending, each of the electrode needles 22 is substantially spaced from the other electrode needles 22. Because RF energy is applied to the affected tissue area interstitially, having substantial spacing between the electrode needles 22 facilitates the spread of RF energy deposition, thereby treating a large volume of tissue. The electrode needles 22 are arranged generally coaxially relative to the tube when the balloon member 18 is in its collapsed configuration and its expanded configuration.

With reference to FIG. 5, the multi-lumen tube 14 includes a passageway 26 for receiving air or liquid, either of which can be used to inflate the balloon member 18 (see FIG. 3) to its fully expanded configuration (see FIG. 3). This could be simply done with a syringe. A passageway 28 is also provided for receiving a vacuum to evacuate the air or liquid from the balloon member 18, thereby causing it to deflate and assume its fully collapsed configuration as shown in FIG. 2. This could also be done with a syringe. Alternatively, the multi-lumen tube 14 can employ a single passageway (not shown) that can receive air, fluid, and vacuum, rather than having the two separate passageways 26, 28. Further, a passageway 30 is sized and shaped to allow the wire leads 24 (see FIG. 4) to pass therethrough.

Referring to FIG. 3, the multi-lumen tube 14 includes a vent 32 for receiving air/liquid from the passageway 26 (see FIG. 5), a vent 34 for receiving vacuum from the passageway 28 (see FIG. 5), and a plurality of wire vents 36, each of which is sized and shaped to allow one of the wire leads 24 to pass therethrough and connect to one of the electrode needles 22. The vents 32, 34 and the wire vents 36 are located underneath the balloon member 18.

When inflated as shown in FIG. 3, the balloon member 18 has a substantially cylindrically-shaped configuration and includes an interior chamber 38 filled with air or liquid. The balloon member 18 can be made from a material which is selected from a group including silicone, latex, urethane, and other flexible polymers.

In operation, a conventional laparoscopic trocar (not shown) is initially placed through the skin. The RF device 10 is then applied through the laparoscopic trocar such that the RF device 10 enters the open body cavity. Note that in the foregoing step, the multi-lumen tube 14 is in its retracted position (see FIG. 1).

As shown in FIG. 2, the multi-lumen tube 14 is then extended from the outer sheath 12. Turning now to FIG. 3, the balloon member 18 is fully inflated with air or liquid so as to assume its expanded configuration. As the balloon member 18 inflates, the electrode needles 22 move toward their deployed configuration. The array of electrode needles 22 is now placed into the target tissue. Voltage is then supplied to the electrode needles 22 such that RF energy is emitted therefrom to the tissues surrounding each of the electrode needles 22. After a predetermined time period between 5 and 50 minutes, preferably 15 minutes, the power to the electrode needles 22 is terminated. Next, the RF device 10 is removed from the target tissue and then from the body cavity by initially deflating the balloon member 18 into its collapsed configuration such that the electrode needles 22 assume their compressed configuration, and then retracting the multi-lumen tube 14 into the outer sheath 12. Lastly, the device is removed from the body through the trocar.

As is evident from the description above, the present invention provides numerous advantages. For instance, because each of the electrode needles 22 extends in a substantially parallel relationship relative to the other electrode needles 22 as shown in FIG. 3, the heating and electric fields will be homogenous along the length of the electrode needles 22. Only the desired penetration depth is used since the electrode needles 22 can be inserted into tissue between 5 and 50 mm, preferably 30 mm. Further, the RF device 10 provides a more predictable heating than that of competitive devices. The RF device 10 can thermally treat malignant or benign pathologies without requiring surgery.

It should be noted that the RF device 10 can have numerous modifications and variations. For instance, the RF device 10 can be either disposable or non-disposable. The RF device 10 can have laparoscopic applications and can be used to treat various sites such as the liver, the lung, and fibroids on or in the wall of the uterus. Also, the RF device 10 can have percutaneous applications and can be used to treat various sites such as the breast and the prostate. The RF device 10 can employ other means, rather than the balloon member 18, to deploy the electrode needles 22. For example, the RF device 10 can employ mechanical means to space the electrode needles 22. The electrode needles 22 can be insulated in sections to regulate the conductive portion and the heating field. In such aspects, RF current will flow only through the needle portion that is not covered by insulation. The RF device 10 can employ alternative energy sources such as laser fibers, ultrasound PZT based cylinders, chemical, microwave antennas, and/or a cryogenic device. The foregoing energy sources can include either catheters or needles. All such variations and modifications are intended to be included within the scope of the invention as defined in the appended claims.

What is claimed is:

1. A medical device used for thermal treatment of tissue, comprising an elongated tube having a longitudinal axis; an expandable member attached to said elongated tube, said expandable member being expandable from a collapsed configuration to an expanded configuration; and a plurality of energy-emitting electrodes attached to said expandable member such that each of said electrodes is arranged generally coaxially relative to said tube when said expandable member is in its said collapsed and expanded configurations.

2. The medical device of claim 1, further comprising a sheath sized and shaped so as to receive said tube and said expandable member when said expandable member is in its said collapsed configuration.

3. The medical device of claim 2, wherein each of said electrodes moves radially outward from said tube as said expandable member moves from its said collapsed configuration to its said expanded configuration.

4. The medical device of claim 3, wherein said electrodes are in close proximity to each other when said expandable member is in its said collapsed configuration and wherein said electrodes are spaced apart from each other when said expandable member is in its said expanded configuration.

5. The medical device of claim 4, wherein said tube extends completely through said expandable member, whereby said expandable member is positioned along an intermediate length of said tube.

6. The medical device of claim 5, wherein each of said electrodes has a generally linear shape.

7. The medical device of claim 6, wherein said tube is movable between a retracted position, in which said tube retracts into said sheath, and an extended position, in which said tube extends axially outward from said sheath.

8. The medical device of claim 7, wherein said tube includes a plurality of channels, at least one of said channels being sized and shaped so as to allow a fluid to pass therethrough.

9. The medical device of claim 8, wherein said expandable member is expandable to its said expanded configuration when a fluid is passed through said at least one of said channels.

10. The medical device of claim 9, wherein said expandable member is collapsible to its said collapsed configuration when a vacuum is supplied to said expandable member through at least another of said channels.

11. The medical device of claim 10, further comprising a plurality of wire leads connected to said electrodes.

12. The medical device of claim 11, wherein at least another of said channels is sized and shaped to allow said wire leads to pass therethrough.

13. The medical device of claim 12, wherein said expandable member is a balloon member.

14. The medical device of claim 13, wherein said electrodes emit RF energy.

15. The medical device of claim 14, wherein said balloon member is substantially cylindrically-shaped when it is in its said expanded configuration.

16. The medical device of claim 15, wherein said electrodes surround said tube when said balloon member is in its expanded configuration.

17. The medical device of claim 16, wherein said expandable member is attached to said tube by a solvent-based glue.

18. A method for performing thermal treatment of tissue using a medical device which includes a tube, an expandable member attached to the tube and expandable from a collapsed configuration to an expanded configuration, and a plurality of electrodes attached to the expandable member, said method comprising the steps of:
(a) inserting the medical device within a tissue area with the expandable member in its collapsed configuration, in which the electrodes are in close proximity to each other and in which all of the electrodes are arranged generally coaxially relative to the tube; and
(b) expanding the expandable member so that it assumes its expanded configuration, in which the electrodes are spaced apart from each other and, in which all of the electrodes are arranged generally coaxially relative to the tube.

19. The method of claim 18, wherein the method is used in a laparoscopic application.

20. The method of claim 19, wherein the method is used in a percutaneous application.
